(19) United States
(12) Patent Application Publication
(10) Pub. No.: US 2024/0065983 A1
(43) Pub. Date: Feb. 29, 2024

(54) COMPOSITION AND METHOD FOR TREATING COVID-19

(71) Applicant: University of South Florida, Tampa, FL (US)

(72) Inventors: Subhra Mohapatra, Lutz, FL (US); Shyam S. Mohapatra, Lutz, FL (US); Karthick Mayilsamy, Tampa, FL (US); Eleni Markoutsa, Tampa, FL (US); Andrew McGill, Tampa, FL (US)

(21) Appl. No.: 18/384,528

(22) Filed: Oct. 27, 2023

Related U.S. Application Data
(63) Continuation of application No. PCT/US2022/026635, filed on Apr. 28, 2022.
(60) Provisional application No. 63/201,397, filed on Apr. 28, 2021.

(51) Int. Cl. A61K 9/51; A61K 31/4439; A61K 31/7105; A61P 31/14

(52) U.S. Cl. CPC A61K 9/5123; A61K 31/4439; A61K 31/7105; A61P 31/14

(57) ABSTRACT

A multi-functional broad-spectrum antiviral and anti-inflammatory nanosystem and methods of treating, including prophylactically, coronavirus infections, such as those caused by severe acute respiratory syndrome coronavirus 2 (SARS-CoV-2), by administering such nanosystem to a patient is presented. The nanosystem may be comprised of a combination of therapeutic agents directed to the particular coronavirus encapsulated in a nanoparticle that is surface coated with a targeting moiety. For CoV-2 infections, an antiviral such as the PPAR-γ agonist leriglitazone (LG) and an siRNA targeting a conserved sequence of the virus can be encapsulated within a nanoparticle surface coated with a fatty acid such as linoleic acid, as the targeting moiety. Administration can occur intranasally prior to infection for prophylactic treatment or post-infection for treatment of the viral infection.

### CROSS REFERENCE TO RELATED APPLICATIONS

This application is continuation of and claims priority to International Patent Application No. PCT/US2022/026635, entitled “Composition and Method for Treating Covid-19”, filed Apr. 28, 2022 which is a nonprovisional of and claims priority to U.S. Provisional Patent Application No. 63/201,397, entitled “A Broad Antiviral Anti-inflammatory Nanosystem”, filed Apr. 28, 2021, the contents of each of which are hereby incorporated by reference into this disclosure.

### FIELD OF INVENTION

This invention relates to treatment and/or prevention of respiratory viruses including coronaviruses. Specifically, the invention provides a method of preventing and/or treating severe acute respiratory syndrome coronavirus 2 (SARS-CoV-2) using a novel nanosystem which incorporates an antiviral with an siRNA into a nanoparticle that is surface coated with a long-chain fatty acid(s) such as linoleic acid as a targeting moiety.

### BACKGROUND OF THE INVENTION

The recent emergence of Severe Acute Respiratory Syndrome Virus-2 (SARS-CoV-2, synonym CoV2) in December of 2019 in Wuhan, China, and the subsequent declaration of a pandemic by the World Health Organization, reaffirms the clinical significance of emerging coronaviruses. CoV2 has been characterized as a virus causing pneumonia and severe respiratory distress. These severe manifestations of viral infection, particularly burden both the elderly, adults and young with underlying conditions. (Park S E. Epidemiology, virology, and clinical features of severe acute respiratory syndrome-coronavirus-2 (SARS-CoV-2; Coronavirus Disease-19). Clin Exp Pediatr. 2020; 63(4):119-24. Epub 2020/04/07). It has been suggested that CoV2 will continue to remain as a serious agent and add to the repertoire of other seasonal respiratory infections caused by influenza, respiratory syncytial virus (RSV), and rhinovirus. Of note, vaccines and a limited number of moderately effective therapies including dexamethasone, remdesivir and molnupiravir are available against CoV2. (McGill A R, Kahlil R, Dutta R, et al. SARS-CoV-2 Immuno-Pathogenesis and Potential for Diverse Vaccines and Therapies: Opportunities and Challenges. Infect Dis Rep. 2021; 13(1):102-25. Epub 2021/02/10). Monoclonal antibody therapy has also presented some success, however the therapy must be administered within 7 days of the onset of symptoms and is most effective on mild to moderate cases of COVID-19. Unfortunately, however, about one-third of the US population does not practice adequate public health measures such as using face masks or getting vaccinated. Hence, there is a dire need to develop additional broad anti-viral agents against respiratory viral infections with pandemic potential.

SARS-CoV-2 and other zoonotic coronaviruses (CoVs) cause devastating disease and remain uncontrolled. During the past 20 years, five novel CoVs have emerged to cause severe illness in new hosts, three of which are human respiratory pathogens: severe acute respiratory syndrome (SARS)-CoV (2002-2004), Middle East respiratory syndrome (MERS)-CoV (2012-present), and SARS-CoV-2 (2019-present). (McGill A R, Kahlil R, Dutta R, et al. SARS-CoV-2 Immuno-Pathogenesis and Potential for Diverse Vaccines and Therapies: Opportunities and Challenges. Infect Dis Rep. 2021; 13(1):102-25. Epub 2021/02/10). Viruses similar to SARS-CoV, MERS-CoV, and SARS-CoV-2 have been found in reservoir species such as bats and pangolins. (Wacharapluesadee S, Tan C W, Maneeorn P, et al. Evidence for SARS-CoV-2 related coronaviruses circulating in bats and pangolins in Southeast Asia. Nature communications. 2021; 12(1):972. Epub 2021/02/11). Thus, in addition to the recent pandemic that has killed thus far ˜6 million globally including ˜1 million Americans, all evidence indicates that new CoV emergence will continuously threaten global health at the animal-to-animal, animal-to-human, and human-to-human interfaces. Potent, broad-spectrum, resistance-averting, and mucosally-administered antivirals are thus urgently needed to treat CoV2, which would allow rapid response to future high-consequence zoonotic CoVs in form of prophylactics and/or therapeutics.

Given the lack of available treatments for COVID-19, what is needed is a composition that is efficacious as a treatment and/or preventative for infection by SARS-CoV-2 virus.

### SUMMARY OF INVENTION

The inventors found that compositions combining broadly antiviral and anti-inflammatory entities integrated in a nano drug delivery system are one of the most effective approaches to mitigate severity of respiratory viral infections thus decreasing mortality and morbidities.

In an embodiment, a multi-functional broad-spectrum antiviral and anti-inflammatory nanosystem is presented comprising at least one nanoparticle surface coated with a targeting moiety, which targets a coronavirus, to form a targeted nanoparticle and at least one therapeutic agent encapsulated within the at least one targeted nanoparticle.

The coronavirus may be severe acute respiratory syndrome coronavirus 2 (SARS CoV-2) virus and the targeting moiety may be targeted to this virus. The targeting moiety may be a fatty acid selected from the group consisting of linoleic acid (LLA) and linolenic acid (LNA).

The at least one therapeutic agent may be a peroxisome proliferator activated receptor gamma (PPAR-γ) agonists, such as leriglitazone (LG) or pioglitazone (PG) or other metabolites of pioglitazone; an siRNA targeting conserved regions of the SARS CoV-2 virus, such as siUTR, siLPro, or siMPro; and combinations thereof. In some embodiments, a combination of therapeutic agents is used for example, LG combined with one or more siRNA(s).

In another embodiment, a method of treating a coronavirus infection in a patient in need thereof is presented comprising: administering to the patient in need thereof a therapeutically effective amount of a composition comprising nanoparticles with their surface coated with at least one targeting moiety to form a targeted nanoparticle; at least one therapeutic agent encapsulated within the at least one targeted nanoparticle, wherein the at least one therapeutic agent is a peroxisome proliferator activated receptor gamma (PPAR-γ) agonist, an siRNA targeting a conserved sequence in the coronavirus, or combinations thereof; and a pharmaceutically acceptable carrier. The administration of the composition may be intranasally and may reduce viral replication in coronavirus-infected cells of the patient.

The coronavirus may be severe acute respiratory syndrome coronavirus 2 (SARS CoV-2) virus. The targeting moiety may target a coronavirus, specifically the SARS CoV-2 virus and may be a medium or long chain fatty acid selected from the group consisting of linoleic acid (LLA) and linolenic acid (LNA).

The PPAR-γ agonist may be leriglitazone (LG) or pioglitazone (PG) and the siRNA may be siUTR, siLPro, or siMPro. In some embodiments, the at least one therapeutic agent is a combination of LG and an siRNA.

In a further embodiment, a method of preventing a coronavirus severe acute respiratory syndrome coronavirus 2 (SARS CoV-2) infection in a patient in need thereof is presented comprising: prophylactically administering to the patient in need thereof a therapeutically effective amount of a composition, the composition comprising nanoparticles surface coated by at least one targeting moiety to form a targeted nanoparticle; at least one therapeutic agent encapsulated within the at least one targeted nanoparticle, wherein the at least one therapeutic agent is a peroxisome proliferator activated receptor gamma (PPAR-γ) agonist, an siRNA targeting a conserved sequence in the coronavirus, or combinations thereof; and a pharmaceutically acceptable carrier. The prophylactic administration may be intranasally and serves to inhibit the coronavirus infection.

The coronavirus may be severe acute respiratory syndrome coronavirus 2 (SARS CoV-2) virus. The targeting moiety may target the severe acute respiratory syndrome coronavirus 2 (SARS CoV-2) virus and be a fatty acid selected from the group consisting of linoleic acid (LLA) and linolenic acid (LNA).

The PPAR-γ agonist may be leriglitazone (LG) or pioglitazone (PG) and the siRNA may be siUTR, siLPro, or siMPro. In some embodiments, the at least one therapeutic agent is a combination of LG and an siRNA.

### BRIEF DESCRIPTION OF THE DRAWINGS

For a fuller understanding of the invention, reference should be made to the following detailed description, taken in connection with the accompanying drawings, in which:

FIG. 1A-C are a series of images depicting docking of PG/LG with spike trimer of CoV-2. A) Molecular docking of Spike protein trimer (6VYB) overlaid onto Spike/ACE2 Interface (6LZG); Pose 1, S=−6.06038; RMSD=5.513527; E-Conf=−82.4773 and % C=86.18. B & C) Molecular docking with mutant spike proteins, (B) K417N mutant and (C) N501Y mutant.

FIG. 1D-E are a series of images depicting docking of PG/LG with spike trimer of CoV-2. D & E) Molecular docking with mutant spike proteins, (D) triple mutant and (E) E484K mutant.

FIG. 1F-G are a series of images depicting docking of PG/LG with spike trimer of CoV-2. F) graph depicting interference of RBD-ACE2 interaction by LG was assayed using SARS-CoV-2 surrogate virus neutralization test kit (Genscript), n=4 p≤0.05. Anti-RBD was used as a positive control. G) LG inhibits infection of DBT mouse cells infected by a mouse coronavirus MHV-A59-GFP. Upper panel: brightfield (BF), lower panel merged images of BF and GFP shown.

FIG. 2A-B are a series of graphs depicting broad spectrum anti-viral and anti-inflammatory activity of LG. Histograms showing mRNA expression assessed by qPCR. A) Effect of LG on poly I:C (P I:C) stimulated Calu 3 cells with or without LG treatment, B) Anti-inflammatory effect of LG treatment in NL63 infected Calu 3 cells. Cells were collected 48 hrs of post infection (PI) with NL63 virus (0.1 MOI) (C, Control) and treated with LG (20 uM) (T, Treatment), n=3, Data expressed as mean±SEM, * Compared to NL63, *p<0.05, **p<0.005, ***p<0.0005.

FIG. 3A-B are a series of images depicting dose-dependent anti-viral activity of LG. A) LG treatment decreases SARS CoV2 infection in Caco2 cells. The Caco 2 cells were infected with 0.5 MOI of CoV2-GFP and 24 h PI treated with LG (10-40 uM). At 48 h PI cells were fixed and stained with DAPI. B) Histogram represents Intden/unit area. Mock—UV inactivated virus, n=2, Data expressed as mean±SEM, * Compared to control, ^{#} compared to Mock.

FIG. 4A-B are a series of graphs depicting LG decreases expression of both N and S viral transcripts and inflammatory genes across CoV2 variants. Caco-2 were infected with 0.1 MOI of CoV2 variants and 24 h post infection, cells were treated with LG (20 uM). WA: Washington strain, UK: UK variant and SA: South African variant. 48 h post infection, A) N and S RNA and B) cytokines were detected by qPCR.

FIG. 4C-F are a series of graphs depicting LG decreases expression of both N and S viral transcripts and inflammatory genes across CoV2 variants. Caco-2 were infected with 0.1 MOI of CoV2 variants and 24 h post infection, cells were treated with LG (20 uM). WA: Washington strain, UK: UK variant and SA: South African variant. 48 h post infection, (C-D) The GFP signals (from CoV2-GFP infected WA strain) were imaged and quantitated using ImageJ, C) images represent plaques of (10⁻⁶) serial diluted for both WA and Delta strain infected samples D); (E-F) Histogram representing quantification of gene expression from SARS-CoV-2 delta infected and LG treated Caco2 cells—N protein expression E), and cytokines F) IL-6, TNF-α, PPARγ, NFkB, CCL20, CCR6 and IL-17, assessed by qPCR, n=4, Data expressed as mean±SEM, # compared to Mock, * compared to Control or B.PLGA, ^{#,}*p<0.05, ^{##,}**p<0.005, ^{###,}***p<0.0005.

FIG. 5A-D are a series of images depicting PLGA-LG decreases infection on MA10-SARS-CoV-2 infected Balb\c Mice: A) Timeline of mice infection study. B) Percentage change in body weight after infection and treatment. C, D) Plaque assay from MA10 infected lung tissues (C) images represent plaques of (10⁻³) serial diluted samples, (D) Histogram representing quantification of plaque assay (pfu/g).

FIG. 5E-F are a series of images depicting PLGA-LG decreases infection on MA10-SARS-CoV-2 infected Balb\c Mice: E) Histogram representing quantification of gene expression from SARS-CoV-2 MA-10 infected lung tissues—N expression, IL-6, TNF-α, NFkB, CCL20, CCR6 and IL-17, assessed by qPCR. F) Histology showing pathology of infected lung tissues, PAS staining (upper panel) and H&E staining (lower panel), scale bar 50μ, inset 10μ. n=4. Data expressed as mean±SEM, # compared to Mock, * compared to Control or B. PLGA, ^{#,}*p<0.05, ^{##,}**p<0.005, ^{###,}***p<0.0005.

FIG. 6A-B is an image depicting a schematic showing A) genome organization of Coronaviridae and selected siRNA targets from the conserved regions of the genome including UTR, PLpro and Mpro, and B) an experimental strategy to test for synergy with LG in mouse coronavirus model.

FIG. 7A-C is a series of images depicting LG inhibits coronavirus infection of DBT cells. Remedesvir (Rem) was used as a control. A) Fluorescence microscopy; B) quantification of GFP florescence intensity; C) N mRNA by qPCR. ****p<0.0005.

FIG. 8A-C are a series of images depicting combination treatment with siUTR and LG shows increased inhibition of viral infection A-B), MHV-N C).

FIG. 8D-E are a series of images depicting combination treatment with siUTR and LG shows increased inhibition of IL-6 D) and TNFα E) gene expression. ***p<0.005, ****p<0.0005.

FIG. 9A-B are a series of images depicting A) crystal structures of PDB6M17 (RBD: ACE:B⁰ AT1); B) Molecular docking positions of fatty acids such as LLA, Oleic, Lauric (LA) and LNA with the respective docking scores are shown.

FIG. 9C-D are a series of images depicting linoleic acid (LLA) as a targeting moiety for BAAN. C) TEM images of Control NPs and LLA-NPs in the presence of NL63 (blue arrows: NPs red arrows: NL63). D) LLA-incorporated liposomal NPs interact with NL63 virus and trigger the release of liposome loaded payloads.

FIG. 9E is a series of images depicting E) LLA exposed cells decreased N gene expression. Calu3 cells were incubated with 200 uM of LLA prior to infection with 0.1 MOI icSARS-CoV-2nMG and cells were imaged at 48 hours post infection. LLA-liposomal NPs (L) but not control NPs (C) significantly decreased viral infection and N gene expression. *p<0.05, ^{##,}**p<0.005, ^{###,}***p<0.0005.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention.

### Definitions

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

As used herein, the singular forms “a,” “an” and “the” include plural referents unless the context clearly dictates otherwise. For example, “a nanoparticle” includes “nanoparticles” or “plurality of nanoparticles”.

As used in this specification and the appended claims, the term “or” is generally employed in its sense including “and/or” unless the context clearly dictates otherwise.

All numerical designations, such as pH, temperature, time, concentration, and molecular weight, including ranges, are approximations which are varied up or down by increments of 1.0, 0.1, 0.01 or 0.001 as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

As used herein, the term “comprising” is intended to mean that the products, compositions, and methods include the referenced components or steps, but not excluding others. “Consisting essentially of” when used to define products, compositions, and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

As used herein, “about” means approximately or nearly and in the context of a numerical value or range set forth means±15% of the numerical.

As used herein “patient” is used to describe a mammal, preferably a human, to whom treatment is administered, including prophylactic treatment with the compositions of the present invention. “Patient” and “subject” are used interchangeably herein.

As used herein “animal” means a multicellular, eukaryotic organism classified in the kingdom Animalia or Metazoa. The term includes, but is not limited to, mammals. Non-limiting examples of mammals include humans, rodents, aquatic mammals, domestic animals such as dogs and cats, farm animals such as sheep, pigs, cows and horses. Wherein the terms “animal” or the plural “animals” are used, it is contemplated that it also applies to any animals.

“Administering” or “administration” as used herein refers to the process by which the compositions of the present invention are delivered to the patient. The compositions may be administered in various ways, including but not limited to, orally, nasally, and parenterally.

A “therapeutic agent” as used herein refers to a substance, component or agent that has measurable specified or selective physiological activity when administered to an individual in a therapeutically effective amount. Examples of therapeutic agents as used in the present invention include antivirals, such as PPARγ agonists leriglitazone, pioglitazone and other metabolites of pioglitazone, and siRNA of CCL20/CCR6. In embodiments where CoV-2 infection is being treated or prevented, the siRNA include, but are not limited to, sequences targeting the 5′ UTR, NSP3, and NSP5 genes such as siUTR, siLPro, and siMPro. At least one therapeutic agent is used in the compositions of the present invention, however in some embodiments, multiple therapeutic agents are used. In some embodiments, one or more therapeutic agents may be encapsulated within a nanoparticle.

A “therapeutically effective amount” as used herein is defined as concentrations or amounts of components which are sufficient to effect beneficial or desired clinical results, including, but not limited to, any one or more of treating symptoms of coronaviruses, particularly CoV-2 infection and preventing coronavirus infection, particularly CoV-2 infection. Compositions of the present invention can be used to effect a favorable change in the condition whether that change is an improvement, such as stopping, reversing, or reducing CoV-2 infection, or a complete elimination of symptoms due to CoV-2 infection. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of the animal and the route of administration. The dose may be adjusted according to response.

The amount of the compound in the drug composition will depend on absorption, distribution, metabolism, and excretion rates of the drug as well as other factors known to those of skill in the art. Dosage values may also vary with the severity of the condition to be alleviated. The compounds may be administered once, or may be divided and administered over intervals of time. It is to be understood that administration may be adjusted according to individual need and professional judgment of a person administrating or supervising the administration of the compounds used in the present invention.

The dose of the compounds administered to a subject may vary with the particular composition, the method of administration, and the particular disorder being treated. The dose should be sufficient to affect a desirable response, such as a therapeutic or prophylactic response against a particular disorder or condition. It is contemplated that one of ordinary skill in the art can determine and administer the appropriate dosage of compounds disclosed in the current invention according to the foregoing considerations.

Dosing frequency for the composition includes, but is not limited to, at least about once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day. In some embodiments, the interval between each administration is constant. For example, the administration can be carried out daily, every two days, every three days, every four days, every five days, or weekly. In some embodiments, the administration can be carried out twice daily, three times daily, or more frequent. Administration can also be continuous and adjusted to maintaining a level of the compound within any desired and specified range.

The administration of the composition can be extended over an extended period of time, such as from about a month or shorter up to about three years or longer. For example, the dosing regimen can be extended over a period of any of about 2, 3, 4, 5, 6, 7, 8, 9, 10, 11, 12, 18, 24, 30, and 36 months. In some embodiments, there is no break in the dosing schedule. In some embodiments, the interval between each administration is no more than about a week.

The compounds used in the present invention may be administered individually, or in combination with or concurrently with one or more other compounds used against viruses, including coronaviruses such as SARS CoV-2. Additionally, compounds used in the present invention may be administered in combination with or concurrently with other therapeutics for coronaviruses or other respiratory viruses.

“Prevention” or “preventing” or “prophylactic treatment” as used herein refers to any of: halting the effects of coronavirus infection, reducing the effects of coronavirus infection, reducing the incidence of coronavirus infection, reducing the development of coronavirus infection, delaying the onset of symptoms of coronavirus infection, increasing the time to onset of symptoms of coronavirus infection, and reducing the risk of development of coronavirus infection. In some embodiments, the coronavirus infection is SARS CoV-2.

“Treatment” or “treating” as used herein refers to any of the alleviation, amelioration, elimination and/or stabilization of a symptom, as well as delay in progression of a symptom of a particular disorder. For example, “treatment” of coronavirus infection may include any one or more of the following: amelioration and/or elimination of one or more symptoms associated with coronavirus infection, reduction of one or more symptoms of coronavirus infection, stabilization of symptoms of coronavirus infection, and delay in progression of one or more symptoms of coronavirus infection. Treatment may include reduction of viral replication in cells and/or reducing inflammation associated with coronavirus infection as shown through reduction in inflammatory cytokine expression. In some embodiments, the coronavirus infection is CoV-2.

“Infection” as used herein refers to the invasion of one or more microorganisms such as bacteria, viruses, fungi, yeast, or parasites in the body of a patient in which they are not normally present. In certain embodiments, the infection is from a respiratory virus such as a respiratory syncytial virus, Influenza virus, or coronavirus. In some embodiments, the coronavirus is severe acute respiratory syndrome coronavirus 2 (SARS-CoV-2). Other coronaviruses contemplated herein include, but are not limited to, severe acute respiratory syndrome coronavirus 1 (SARS-CoV-1), Middle East respiratory syndrome coronavirus (MERS-CoV), human coronavirus OC43 (HcoV-OC43), human coronavirus 229E (HcoV-229E), porcine deltacoronavirus (PDCoV) (porcine), infectious bronchitis virus (IBV, avian), and other coronaviruses of pandemic potential including Delta coronavirus, duvinacovirus, Embecovirus, Gammacoronavirus, Merbecovirus, Nobecovirus and Sarbecovirus.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pennsylvania, Mack Publishing Company, 19^{th} ed.) describes formulations which can be used in connection with the subject invention.

For ease of administration, the subject compounds may be formulated into various pharmaceutical forms. As appropriate compositions there may be cited all compositions usually employed for systemically or topically administering drugs. To prepare the pharmaceutical compositions of this invention, atranorin or other polyphenolic lichen acid isolate, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for administration nasally, orally, rectally, percutaneously, or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules often represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution.

“Nanoparticle” as used herein refers to a particle or structure which is biocompatible with and sufficiently resistant to chemical and/or physical destruction by the environment of such use so that a sufficient number of the nanoparticles remain substantially intact after delivery to the site of application or treatment and whose size is in the nanometer range. For the purposes of the present invention, a nanoparticle typically ranges between about 1 nm to about 1000 nm, preferably between about 50 nm and about 500 nm, more preferably between about 50 nm and about 350 nm, more preferably between about 100 nm and about 250 nm. As used herein, the term “nanoparticle” includes, but is not limited to, micelles, polymeric nanoparticles, and lipid-based nanoparticles such as liposomes and niosomes. In some embodiments, the nanoparticles have a core structure comprised of PLGA-PVA-chitosan polymers.

“Polymer” as used herein refers to a relatively high molecular weight organic compound, natural or synthetic, whose structure can be represented by a repeated small unit, the monomer. Synthetic polymers are typically formed by addition or condensation polymerization of monomers. A polymer comprised of two or more different monomers is a copolymer. In some embodiments, the polymers used to form the nanoparticles exist in a triblock copolymer of poly(lactic acid-co-glycolic acid) (PLGA)—poly(vinyl alcohol) (PVA)—chitosan. Other polymers are contemplated for use including polyacrylic acid.

“Targeting moiety” as used herein refers to a fatty acid, peptide, aptamer, antibody, protein, carbohydrate, vitamin, or organic small molecule capable of being linked to a nanoparticle and having an affinity for a specific binding partner on a coronavirus viral particle. The targeting ligand is preferably selective as opposed to non-selective. In some embodiments where the coronavirus is SARS CoV-2, the targeting moiety binds to the CoV-2 spike protein or ACE2-spike interface. In some embodiments, the at least one targeting moiety is used and is a fatty acid including, but not limited to, linoleic acid (LLA), linolenic acid (LNA), oleic acid, and lauric acid.

“Therapeutic nanoparticle” as used herein refers to nanoparticles containing at least one therapeutic agent. In some embodiments, the therapeutic nanoparticles contain an siRNA and/or a PPARγ agonist. In some embodiments, the therapeutic nanoparticles have a targeting moiety attached.

“Targeting nanoparticles” or “targeting nanoparticle composition” as used herein refers to at least one therapeutic nanoparticle, optionally suspended in a pharmaceutically acceptable carrier. In some embodiments, the targeting nanoparticles of the instant invention are used to treat coronavirus infections. In some embodiments, the coronavirus infection is a SARS CoV-2 infection.

“Broad-spectrum antiviral and anti-inflammatory nanosystem” or “BAAN” as used herein refers to a composition of targeting nanoparticles containing at least one therapeutic agent that can be used to treat or prevent viral infection and/or inflammation. In some embodiments, the viral infection being treated or prevented is a coronavirus infection. In some embodiments, the coronavirus infection is a SARS CoV-2 infection with the nanoparticles having a targeting moiety of LLA or LNA to bind to the CoV-2 spike protein or ACE2-spike interface. In some embodiments, the therapeutic agent encapsulated is an siRNA and a PPARγ agonist. The siRNA may be at least one sequence selected from sequences targeting the 5′ UTR, NSP3, and NSP5 genes or combinations thereof. The PPARγ agonist may be leriglitazone, pioglitazone, other metabolites of pioglitazone, or combinations thereof. In some embodiments, the BAAN is comprised of a cocktail of siRNAs in combination with LG encapsulated within an LLA-coated nanoparticle.

PPARγ Agonists

Inflammation plays a significant role in symptoms experienced by both acute and long haul COVID-19 patients. SARS CoV-2 infection can lead to uncontrolled inflammation, which in turn leads to a cytokine storm, i.e. an exaggerated release of cytokines in response to infection that is due to unregulated release of pro-inflammatory cytokines such as IL-6, IL-1β, and TNFα, among others.

Discovery of a broad anti-viral and -inflammatory activity has provided a new impetus for novel coronavirus therapy. The CoV-2 pandemic has inspired a molecular docking analysis of FDA approved drugs that bind to the CoV2 Spike protein. The inventors previously found that pioglitazone (PG) had one of the top docking scores. Pioglitazone is a PPAR-γ agonist which has previously been found to inhibit secretion of pro-inflammatory cytokines and increase secretion of anti-inflammatory cytokines. (Qiu D, Li X N. Pioglitazone inhibits the secretion of proinflammatory cytokines and chemokines in astrocytes stimulated with lipopolysaccharide. Int J Clin Pharmacol Ther 2015 September; 53(9):746-52). Specifically, pioglitazone was found to significantly reduce IL-6 and TNFα mRNA expression to attenuate lung injury. (Kutsukake M, Matsutani T, Tamura K, et al. Pioglitazone attenuates lung injury by modulating adipose inflammation. J Surg Res. 2014; 189(2):295-303).

Leriglitazone (LG) is a soluble orally bioavailable metabolite of PG, which was previously reported to possess potent antiviral activity, particularly against certain RNA viruses such as respiratory syncytial virus (RSV). (Mayilsamy K, Markoutsa E, Das M, et al. Treatment with shCCL20-CCR6 nanodendriplexes and human mesenchymal stem cell therapy improves pathology in mice with repeated traumatic brain injury. Nanomedicine: nanotechnology, biology, and medicine. 2020; 29:102247. Epub 2020/07/01; Matthews L, Kleiner D E, Chairez C, et al. Pioglitazone for Hepatic Steatosis in HIV/Hepatitis C Virus Coinfection. AIDS Res Hum Retroviruses. 2015; 31(10):961-6. Epub 2015/07/28; Das M, Wang C, Bedi R, et al. Magnetic micelles for DNA delivery to rat brains after mild traumatic brain injury. Nanomedicine: nanotechnology, biology, and medicine. 2014; 10(7):1539-48. Epub 2014/02/04; Wan C J, Dong L, Lin J, et al., [PPARgamma agonists against respiratory syncytial virus infection in vitro study]. Zhonghua Shi Yan He Lin Chuang Bing Du Xue Za Zhi. 2011; 25(6):480-2; Arnold R, Konig W. Peroxisome-proliferator-activated receptor-gamma agonists inhibit the release of proinflammatory cytokines from RSV-infected epithelial cells. Virology. 2006; 346(2):427-39. Epub 2005/12/07).

Leriglitazone has been found to significantly reduce biomarkers related to inflammation such as matrix metalloproteinase 9, interleukin-18, interleukin-1β, interleukin-1 receptor agonist, and macrophage inflammatory protein 1β. It is a selective peroxisome proliferator-activated receptor γ agonist (PPAR-γ agonist) that is thought to protect neurons and astrocytes by preventing monocyte and microglial activation. Administration of a therapeutically effective amount of leriglitazone can decrease inflammation by reducing the cytokine storm. This decrease in proinflammatory cytokines aids in an anti-inflammatory response that can resolve inflammatory symptoms associated with CoV-2 infection in the respiratory system, as well as symptoms of other body systems, such as joint pain, body aches, and fatigue.

The inventors unexpectedly discovered that LG is efficacious in inhibiting CoV-2 infection. LG was shown to achieve high and controlled exposure in patients with central nervous system showing both safety and efficacy. (Rodriguez-Pascau L, Vilalta A, Cerrada M, et al. The brain penetrant PPARgamma agonist leriglitazone restores multiple altered pathways in models of X-linked adrenoleukodystrophy. Science translational medicine. 2021; 13(596). Epub 2021/06/04). The inventors discovered that LG also docks to the spike trimer suggesting its potential to inhibit spike-ACE2 interaction. As discussed in the Examples below, the inventors found that LG significantly inhibits CoV-2 infection in diverse cell lines. Further, the inventors found that pretreatment of mice with a poly(lactide-co-glycoside) (PLGA)-based LG nanoformulation significantly inhibited viral infection in mouse coronavirus (mouse hepatitis virus (MHV)), K18 hACE2 transgenic mouse, and mouse adapted coronavirus (MA10) infection models.

siRNA

In addition to LG, the inventors discovered the use of siRNAs as prophylactic and therapeutic agents for viral infection. (Markoutsa E, McGill A R, Singer A, et al. A multifunctional nanoparticle as a prophylactic and therapeutic approach targeting respiratory syncytial virus. Nanomedicine: nanotechnology, biology, and medicine. 2021; 32:102325. Epub 2020/11/14; Zhang W, Yang H, Kong X, et al. Inhibition of respiratory syncytial virus infection with intranasal siRNA nanoparticles targeting the viral NS1 gene. Nature medicine. 2005; 11(1):56-62; Bird G H, Boyapalle S, Wong T, et al. Mucosal delivery of a double-stapled RSV peptide prevents nasopulmonary infection. The Journal of clinical investigation. 2014; 124(5):2113-24. Epub 2014/04/20). Similar strategies by industry has led to successful phase I/II clinical trials validating safety and efficacy of siRNAs as drug target. (DeVincenzo J, Lambkin-Williams R, Wilkinson T, et al. A randomized, double-blind, placebo-controlled study of an RNAi-based therapy directed against respiratory syncytial virus. Proceedings of the National Academy of Sciences of the United States of America. 2010; 107(19):8800-5. Epub 2010/04/28). In preliminary studies, using an in vitro mouse coronavirus model, the inventors found that selected siRNAs can significantly inhibit ongoing virus infection. In some embodiments, the inventors have selected LG and siRNAs for development of broad-spectrum antivirals.

Targeted Nanosystem

A nanosystem delivery approach combining RNA and small-molecule therapies provides a new direction to treat CoV-2. Targeting drugs to the alveolar lung remains challenging. (Ventola C L. Progress in Nanomedicine: Approved and Investigational Nanodrugs. P T. 2017; 42(12):742-55. Epub 2017/12/14; Shi J, Kantoff P W, Wooster R, et al. Cancer nanomedicine: progress, challenges and opportunities. Nature reviews Cancer. 2017; 17(1):20-37. Epub 2016/11/12). Nanoparticle (NP)-based approaches based on systemic delivery have not yet reached their full potential. (Metselaar J M, Lammers T. Challenges in nanomedicine clinical translation. Drug Deliv Transl Res. 2020; 10(3):721-5. Epub 2020/03/14; Greish K, Mathur A, Bakhiet M, et al. Nanomedicine: is it lost in translation? Ther Deliv. 2018; 9(4):269-85. Epub 2018/03/03). Specifically, delivering therapeutics to the CoV-2 syncytia in the alveolar lung without affecting neighboring healthy cells in an important organ, such as the lung, remains a dire need. In order to address this need, the inventors examined the use of a nanoparticle targeting moiety. The molecular docking studies led to the identification of fatty acids, such as linoleic acid (LLA), which targets to a binding pocket in spike-Ace2 interaction that is conserved among coronaviruses, suggesting it as a NP-targeting moiety for CoV2 infected cells. Accordingly, the inventors have developed a multifunctional broad-spectrum antiviral and anti-inflammatory nanosystem (BAAN) incorporating siRNAs cocktail and LG as payloads and surface coated LLA as the targeting moiety against CoV2 and other coronaviruses.

The BAAN may be comprised of at least one therapeutic agent, and in some embodiments two or more therapeutic agents encapsulated in a targeted nanoparticle having a surface moiety targeting infected cells. In support of the BAAN nanosystem, the inventors found that LG significantly reduces CoV-2 infection both in vitro and in vivo. The inventors also discovered that siRNAs against coronaviruses can decrease viral replication by interfering with several viral pathways. Given that the siRNAs are directed to viral pathways, there are few off-target effects for the host. Though siRNAs have been translated to clinical trials, siRNA-based therapies remain to be discovered for CoV-2. (DeVincenzo J, Lambkin-Williams R, Wilkinson T, et al. A randomized, double-blind, placebo-controlled study of an RNAi-based therapy directed against respiratory syncytial virus. Proceedings of the National Academy of Sciences of the United States of America. 2010; 107(19):8800-5. Epub 2010/04/28). Further, the inventors conducted molecular docking of fatty acids and discovered linoleic acid (LLA) docks to the spike-Ace2 interface of CoV-2 with potential to inhibit viral fusion and block cellular entry of virus. Beyond this, LLA has shown to possess anti-inflammatory activity and was reported to be safe in rat and human studies. BAAN allows for packaging a combination of at least two therapeutic agents in a core-shell based targeting nanosystem that can be delivered intranasally to be used as a therapeutic, including prophylactic therapy, against CoV-2 and other coronavirus infections.

Surprisingly, the inventors find that a combination of an anti-inflammatory agent, such as the PPAR-γ agonist leriglitazone, with siRNA, encapsulated in a nanoparticle having a targeting moiety such as linoleic acid (LLA) is able to both synergistically treat and prevent CoV-2 infection in in vivo mouse models. Translating this to humans, this combination also suggests a similar synergistic effect on SARS-CoV-2 in humans. Such combination treatment is advantageous in that the therapeutic agents synergistically act together to reduce multiple neurological and inflammatory symptoms caused by CoV-2 infection as well as prevent CoV-2 infection.

The following non-limiting examples illustrate exemplary systems and components thereof in accordance with various embodiments of the disclosure. The examples are merely illustrative and are not intended to limit the disclosure in any way.

### Example 1—LG Inhibits Spike ACE-2 Interaction and Viral Infection

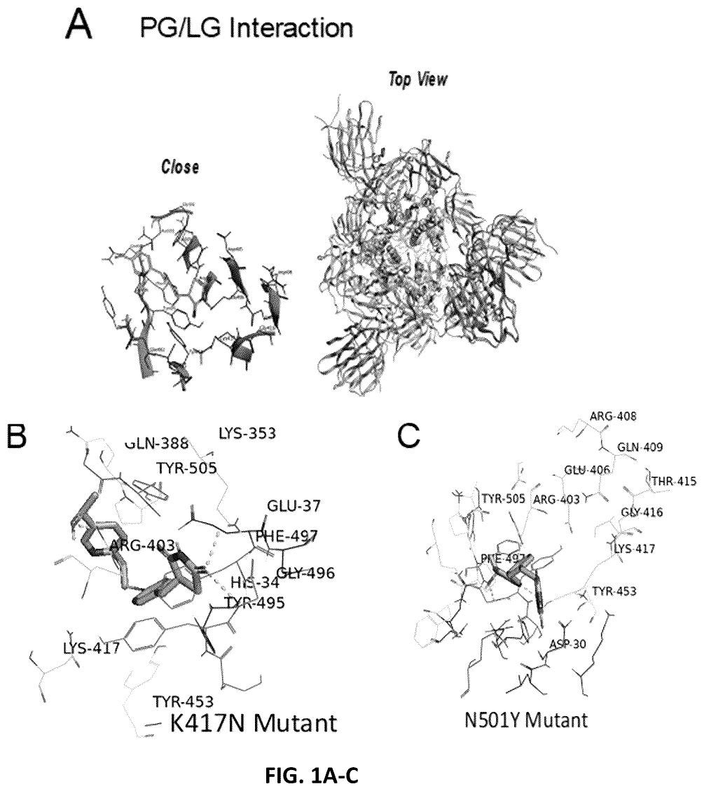

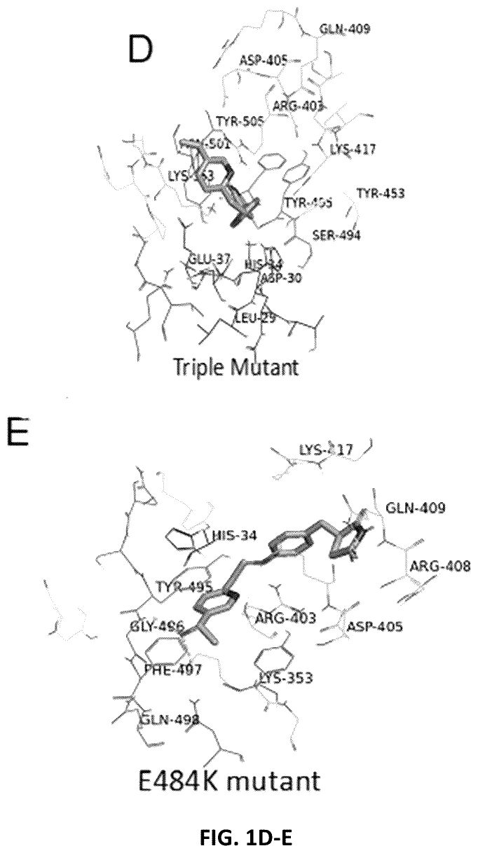

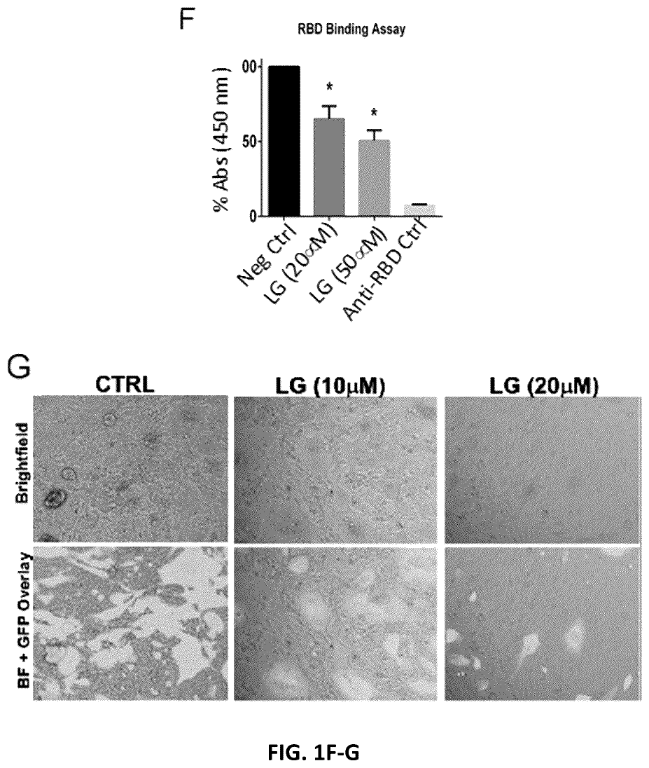

Because of the antiviral properties of PG, it has been suggested that it may have anti-CoV-2 effects, but it is yet to be demonstrated. Also, LG, a PG metabolite with higher solubility than PG, has not been tested for its role in viral infection. Docking of PG/LG to spike protein trimer (6VYB) overlaid onto the spike/ACE2 interface showed relatively high docking affinity on both crystal structures, the top pose exhibiting a docking score of −6.06038 and probability of occurrence % C=86.18% (not shown). Of note, the common acidic thiazolidine-2,4,-dione of PG/LG interacts with specific amino acid residues such as Arg-403 within the binding pocket. Also, Arginine 403 is a part of the integrin (coreceptor for CoV-2 entry)-modulating motif consisting of Arg403-Gly404-Asp405 which has been implicated in initiating viral attachment (FIG. 1A).

These findings demonstrate that LG has the potential to affect viral adhesion and hence entry to host cells. Since the CoV-2 virus has mutated recently in the Arg403 binding pocket, the inventors examined the LG interaction with mutant spike proteins (FIG. 1B-E). The results show that LG can bind to K417N, E484K and N501Y spike mutants that are first detected respectively in beta, delta and omicron CoV2 variants, although a slight nonsignificant shift was found in LG-N501Y binding. To assay the LG interference of Spike-ACE2 interaction, a CoV2 surrogate virus neutralization test kit (Genscript) was used according to manufacturer's instructions. The results showed that LG significantly inhibited the RBD-ACE2 interaction in a dose-dependent manner compared to the negative control (FIG. 1F). To test antiviral activity of LG, the inventors first used mouse DBT cells infected with an engineered mouse coronavirus, MHV-A59-GFP that expresses green fluorescence protein (GFP) in infected cells. The results showed that LG inhibited coronavirus infection in a dose-dependent fashion, 20 uM LG inhibiting >70% infection without killing the cells, as can be seen from the bright field images (FIG. 1G).

### Example 2—LG Exhibits Broad Spectrum Anti-Inflammatory Activity

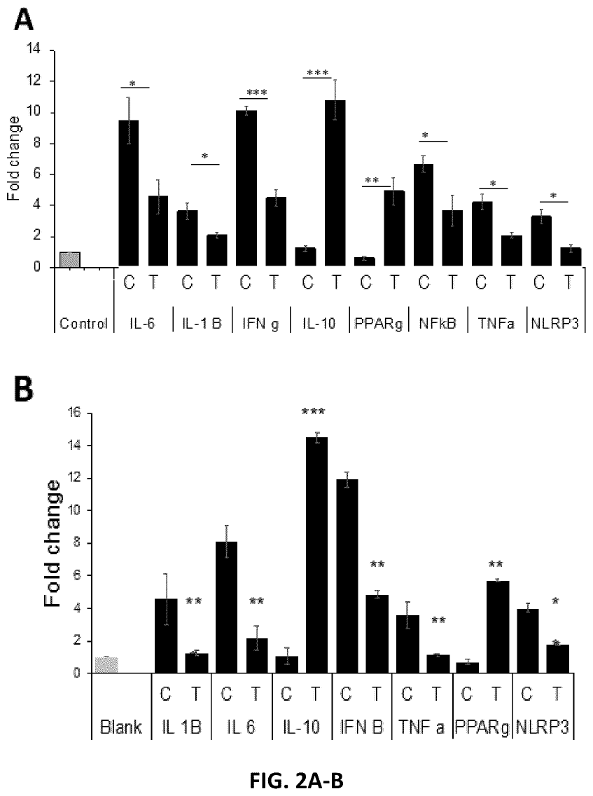

To examine whether LG has broad spectrum antiviral activity, Calu3 cells are stimulated with 10 ug/ml poly IC for 24 h, treated with 20 uM LG for 48 h, and then RNA was isolated for gene expression analyses. The results show that LG inhibits expression of IL-6, IL1β, IFNγ, TNFα, NFkB, and NLRP3 and increases expression of IL-10 and PPARγ(FIG. 2A). Further the inventors examined the effect of LG on the Calu3 lung cells infected with human coronavirus NL63. The results showed altered expression of anti-inflammatory genes at the transcript level similar to that found in poly-IC experiment (FIG. 2B).

### Example 3—LG Exhibits Dose-Dependent Anti-Viral Activity

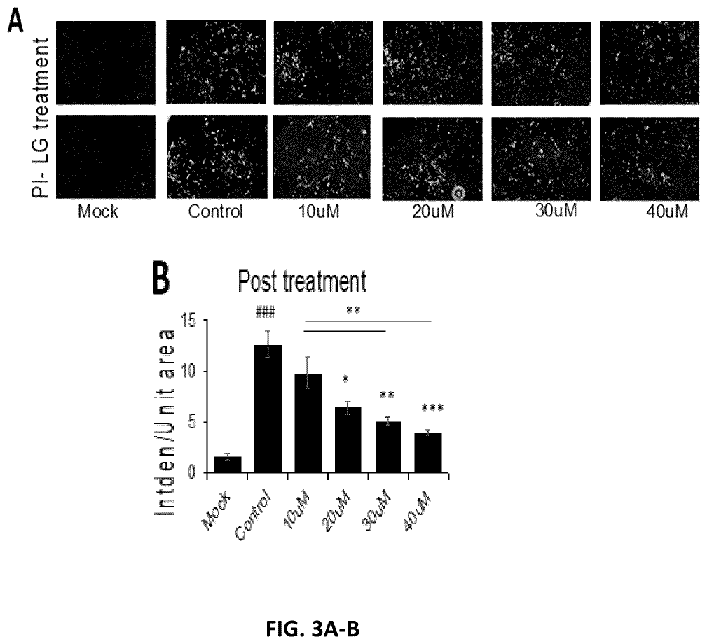

The inventors acquired an infectious cDNA clone of SARS-CoV-2 encoding a stable mNeonGreen reporter icSARS-CoV-2nMG¹⁸ (CoV2-GFP) from Dr. Pei-Yong Shi (Univ Texas Medical Branch) which was expanded and tittered in Vero cells and used to infect Caco2 cells. The inventors examined the cells 24 h post-infection by fluorescent microscopy. These results showed that icSARS-CoV-2nMG infects Caco2 cells that can be tracked easily. The inventors then examined the therapeutic effects of various doses (10 to 40 uM) of LG on CoV2-infected Caco2 cells, and the results show that there is a dose-dependent decrease of CoV2 infection by LG treatment (FIGS. 3A and B).

### Example 4—LG Decreases Expression of Both N and S Viral Transcripts and Inflammatory Mediators Across CoV-2 Variants

The inventors examined the role of LG action in Caco2 cells across different variants of CoV2. Cells were infected with 0.1 MOI SARS CoV2 variants (WA, Washington strain, UK, United Kingdom B.1.1.7, SA, South Africa B.1.351). The infected cells were treated with LG (20 uM) for 48 hours and collected in trizol for RNA extraction.

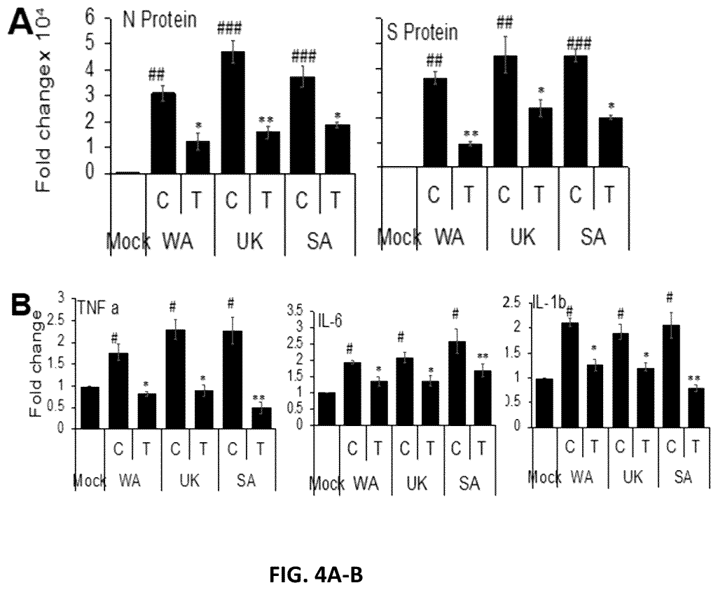

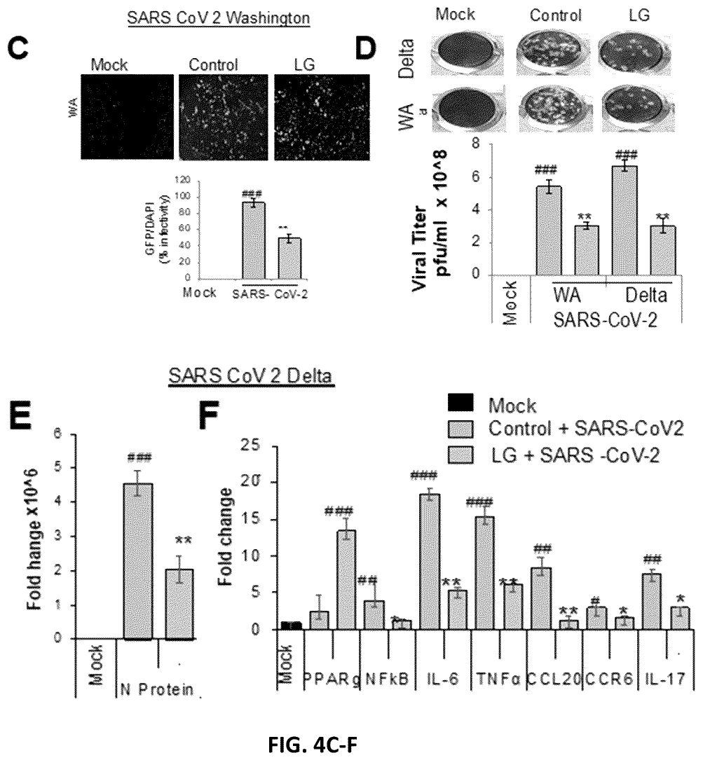

The results showed that LG treatment significantly inhibited the expression of CoV2-N gene involved in replication and of the S genes involved infusion and viral entry for all variants examined including WA, UK, SA (FIG. 4A). The expression inflammatory genes such as TNFα, IL-6 and IL1β, were down-regulated at the transcript level in all CoV2 strains (FIG. 4B). Further, the inventors examined the effect of LG (20 uM) on the WA strain infection of Caco2 cells and the results showed significant reduction in infected GFP-positive cells (FIG. 4C).

The inventors examined LG's antiviral effects for both WA and Delta strains by plaque assay and the results showed that there was >2 log reduction in plaque titers for both strains. (FIG. 4D). Since delta variant causes more severe disease, the effect of LG on this isolate was examined. The results showed that LG decreased expression of N and several inflammatory gene transcripts similar to other strains suggesting potent antiviral effects (FIG. 4E-F). These results are consistent with the antiviral effects seen in some of the other glitazones against RNA viruses, as described previously. (Chojkier M, Elkhayat H, Sabry D, et al. Pioglitazone decreases hepatitis C viral load in overweight, treatment naive, genotype 4 infected-patients: a pilot study. PLoS One. 2012; 7(3):e31516. Epub 2012/03/14; Guerrero C A, Murillo A, Acosta O. Inhibition of rotavirus infection in cultured cells by N-acetyl-cysteine, PPARgamma agonists and NSAIDs. Antiviral Res. 2012; 96(1):1-12; Khattab M, Emad M, Abdelaleem A, et al. Pioglitazone improves virological response to peginterferon alpha-2b/ribavirin combination therapy in hepatitis C genotype 4 patients with insulin resistance. Liver Int. 2010; 30(3):447-54; Omeragic A, Kara-Yacoubian N, Kelschenbach J, et al. Peroxisome Proliferator-Activated Receptor-gamma agonists exhibit anti-inflammatory and antiviral effects in an EcoHIV mouse model. Sci Rep. 2019; 9(1):9428. Epub 2019/07/03; Arnold R, Konig W. Peroxisome proliferator-activated receptor-gamma agonists inhibit the replication of respiratory syncytial virus (RSV) in human lung epithelial cells. Virology. 2006; 350(2):335-46. Epub 2006/04/18).

### Example 5—In Vivo Studies of Effects of LG on SARS CoV-2 in MA10-SARS-CoV-2 Infected Balb\c Mice

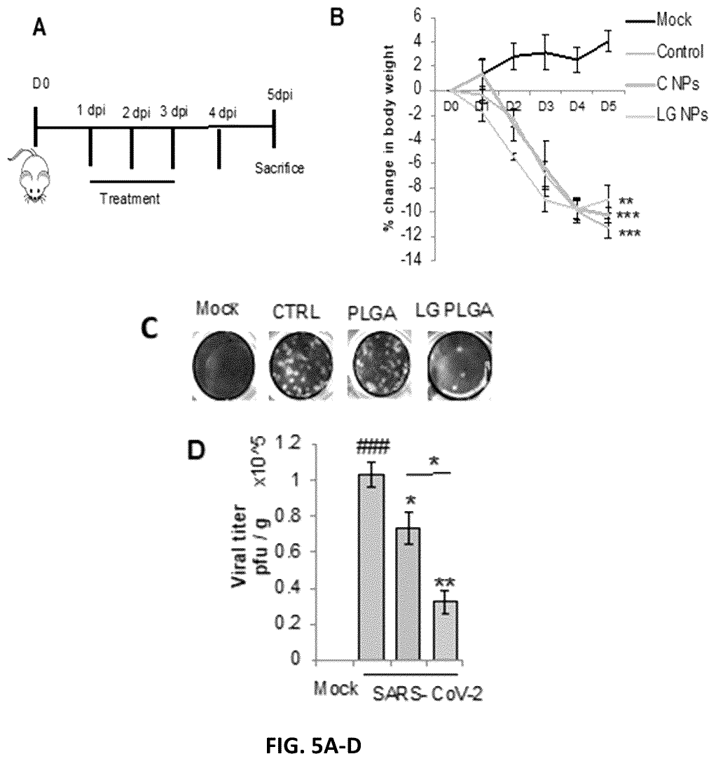

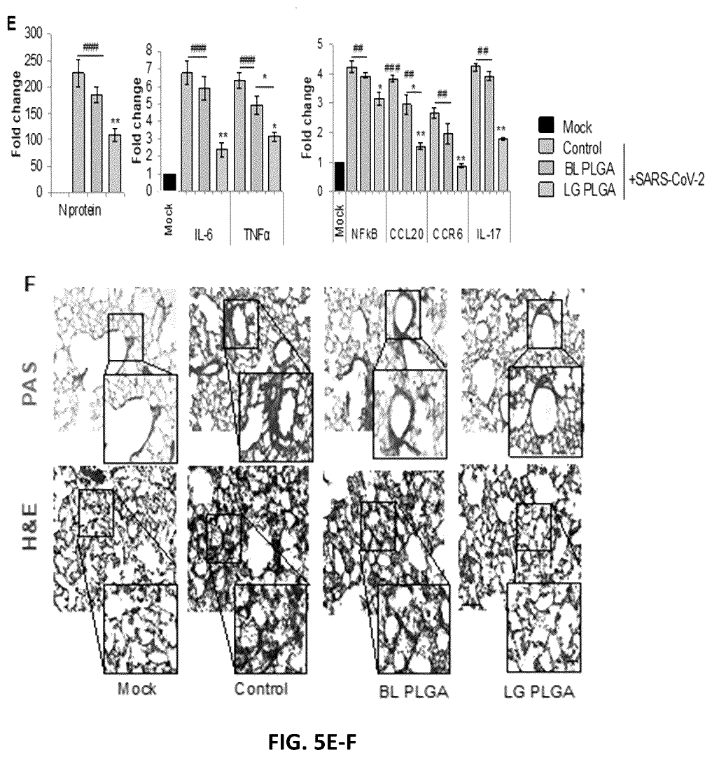

To determine the effect of LG-PLGA NPs in CoV2 infected mice the inventors used the MA10 model described in Kant. (Kant R, Kareinen L, Smura T, et al. Common Laboratory Mice Are Susceptible to Infection with the SARS-CoV-2 Beta Variant. Viruses. 2021; 13(11)). The timeline of mice infection study is shown in (FIG. 5A). Mice were treated with LG packaged in PLGA nanoparticles (LG-NPs) or control NPs (BL-PLGA) starting 24 h pi and mice were sacrificed on day 5 pi (FIG. 5A). The infected lungs show signs of severe virus infection compared to mock infected mice. Percentage change in body weight after infection and treatment was measured and all infected mice showed a ˜10% reduction in body weight during infection period (FIG. 5B). A plaque assay from MA10 infected lung tissues was performed from the lung homogenate and the LG treated mice showed a significant reduction in plaque versus BL-PLGA NP control (FIG. 5C-D). Further, a gene expression analyses from lung RNA after sacrifice was performed by qPCR and the data showed that LG treatment significantly reduced expression of COV2-N, IL-6, TNF-α, NFkB, CCL20, CCR6 and IL-17 (FIG. 5E). Finally, lung histology was performed, and the LG treatment showed reduced pathology as revealed by PAS (mucus production) and H&E staining showing thickening of alveolar septa (FIG. 5F).

### Example 6—siRNA Targets for Coronaviruses

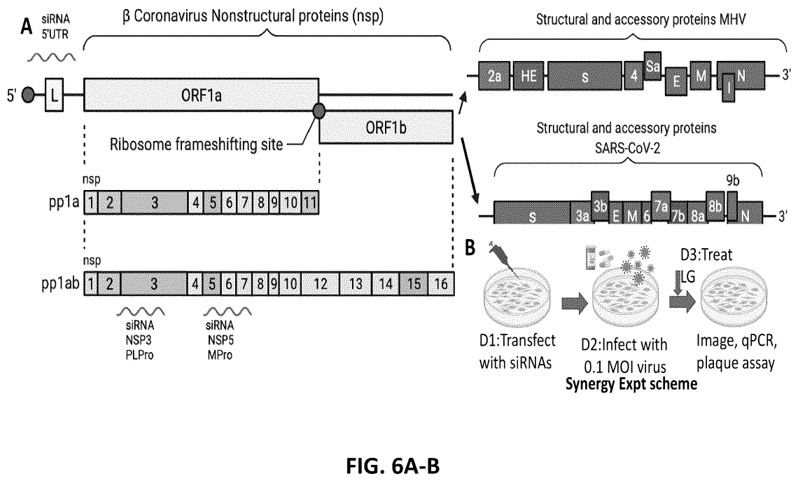

Toward developing a robust broad-spectrum therapy against coronaviruses, the inventors examined the potential of siRNAs with or without LG, with the latter being shown to have >70% efficacy in in vivo studies. The strategy is schematically shown in FIG. 6. Briefly, the inventors compared the coronaviridae genome organization from NCBI genome database and searched for conserved sequences primarily targeting the 5′UTR, NSP3, and NSP5 genes. The sequences at intergenic spacer regions within the positive sense RNA genome were uploaded to InvivoGen siRNA Wizard with a 21nt base limit, while excluding cross reactive mouse sequences. The InvivoGen tool comprised 16-31 sequences per gene and the sequences were selected that have less GC content, as they are conventionally viewed to be more active intracellularly. Selected sequences were then uploaded to IDTDNA, which computationally converted the sequences to RNA, and generated a duplex. siRNA was then resuspended at stock concentrations of 100 nM, diluted at required working stocks, and transfected using lipofectamine 3000 reagent.

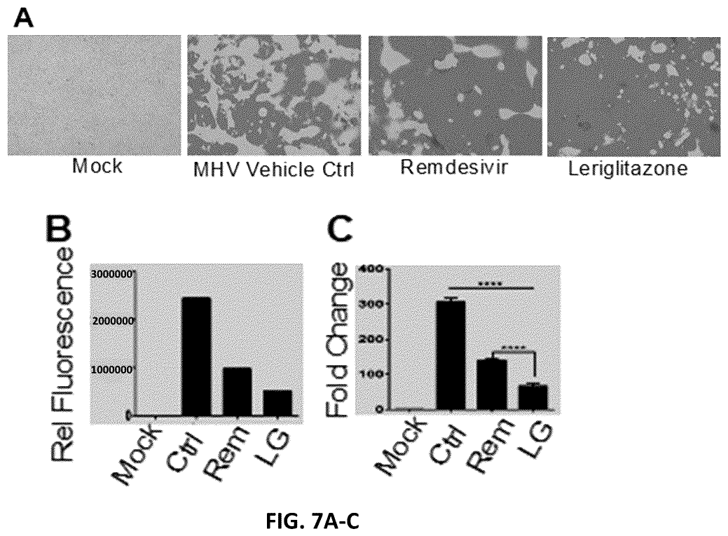

To test the concept of combination therapy, the inventors established a mouse coronavirus model using a MHV-A59-GFP virus that infects DBT (brain cancer cells) wherein cells are readily infected by MHV A59 virus within 24 h pi (FIG. 7A-B). In this model, the treatment of infected cells by LG at 20 uM showed a significant reduction in N transcript when compared to vehicle control. Also, LG showed significantly higher reduction in infection versus Remdesvir (10 nM) used as control (FIG. 7).

Further, to test whether the siRNAs decrease MHV-A59 infection, the inventors tested 4 of the bioinformatically screened siRNAs in in vitro cell cultures using DBT cells and MHV-A59-GFP mouse coronavirus. The results showed that three of the four siRNAs targeting 5′UTR, L-Pro and M-Pro decreased the DBT infection as measured by GFP protein expression (not shown).

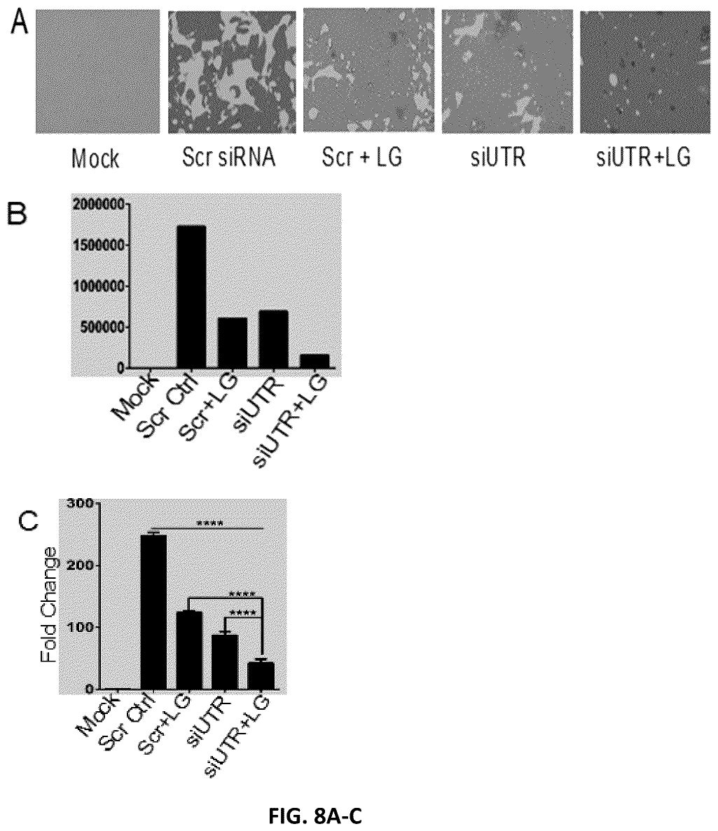

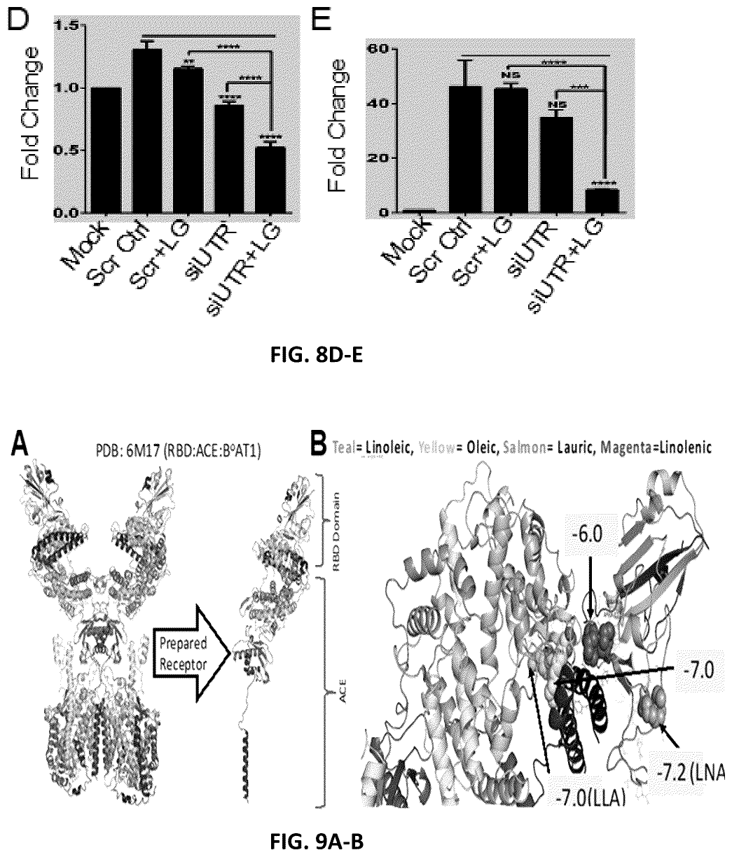

Moreover, the inventors examined whether the siRNAs can be combined with LG, by using siUTR as an example. Thus, DBT cells were transfected with siUTR siRNA or scrambled control siRNA (3 nM) using Lipofectamin 3000 and infected after 24 h and were treated with LG (20 uM) after a further 24 h. After 24 h of LG treatment, cells were imaged, and RNA was isolated from cell to examine viral replication and cytokine secretion. The results showed that cells which received siUTR RNA and LG treatment showed the highest reduction in the expression of MHV N transcript and the expression of cytokine genes such as IL6 and TNFα (FIG. 8A-E).

### Example 7—Linoleic Acid (LLA) as a Targeting Moiety

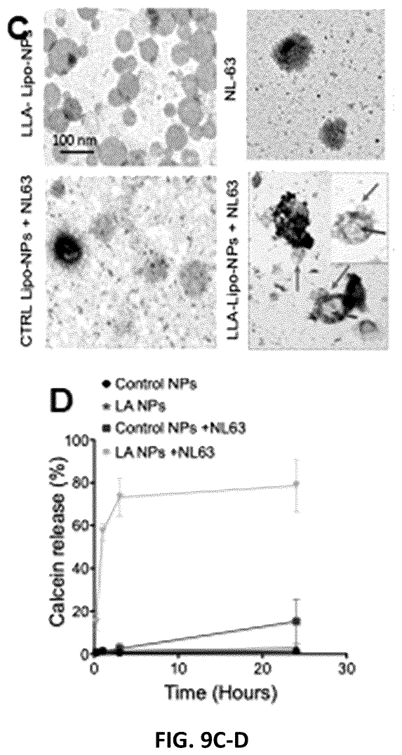

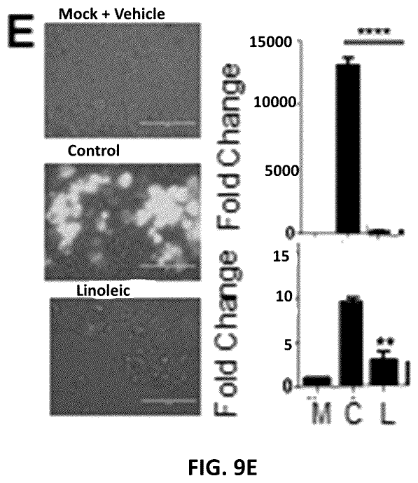

Our molecular docking of medium- and long-chain fatty acids with CoV2-S protein using Molecular Operating Environment and Vina-dock programs led to identification of LLA and Linolenic acid (LNA) interacting with CoV-2 spike protein or ACE2-spike interface (FIG. 9A-B), with docking scores of 7.0 and 7.2, respectively. LLA was selected as a candidate for use as a targeting moiety because: i) it was a top scorer in our docking analyses, ii) it was shown that it binds to CoV2-S protein in three composite binding pockets and this interaction results in a locked S conformation that inhibits the ACE2 from binding to S protein²⁵, iii) this binding pocket is conserved for seven other coronaviruses, iv) it plays an important role in inflammation and immune modulation, and v) the inventors found by TEM analyses that LLA-liposomal NPs show high affinity for the viral surface (FIG. 9C). Also, LLA-lipo-NPs were found to interact with NL63-CoV (FIG. 9D), which caused accelerated particle degradation and calcein release. Moreover, to examine effect LLA on viral infection, Calu3 infected with 0.1 MOI icSARS-CoV-2nMG Incubated 16 hours before 200 uM of LLA and cells were imaged at 48 hours post infection and lysed. The results showed that LLA significantly decreased viral infection as seen by N gene expression (FIG. 9E).

### Example 8—Treatment of CoV-2 Infection with BAAN Nanosystem Containing Both LG and siRNA (Prophetic)

A 49 year old male patient presents with headache, vomiting, nausea, and loss of taste and smell. A diagnosis of CoV-2 infection is confirmed. The patient is intranasally administered a therapeutically effective amount of a BAAN nanosystem composition comprising a plurality of targeted LLA-surface coated nanoparticles encapsulating the combination of the PPARγ agonist LA and siUTR for a time period sufficient to alleviate the symptoms. The patient is retested twice over a several week timespan and tests negative for the virus.

### Example 9—Prophylactic Treatment of CoV-2 Infection with BAAN Nanosystem Containing Both LG and siRNA (Prophetic)

A 38 year old female patient tests negative for COVID-19 and is administered a therapeutically effective amount of a BAAN nanosystem composition comprising a plurality of targeted LLA-surface coated nanoparticles encapsulating the combination of the PPARγ agonist LA and siUTR as a preventative to infection by the CoV-2 virus. The female is exposed to the CoV-2 virus through contact with multiple people infected with the virus. The female does not develop a CoV-2 infection as confirmed by testing.

### CONCLUSION

The inventors have discovered that i) The PLGA-PVA-chitosan polymers together provide a unique core-shell scaffold for developing multifunctional nanosystem to deliver broad-spectrum antiviral and anti-inflammatory drugs such as siRNA and LG to combat the viruses at the nasal site and the lung, where these viruses multiply and cause the disease. ii) LG can exert broad antiviral and anti-inflammatory activities. iii) siRNAs can be combined with LG to provide better anti-viral activities. iv) LLA can be used as a targeting moiety for coronaviruses. v) The inventors have established four important models, a MHV model, a NL63 model, K18-hACE2 model, a CoV2-MA10 mouse model and therapeutics for coronaviruses, especially for preclinical evaluation of BAAN. vi) Also, the inventors have modified our FiSS platform to develop an in-house air-liquid culture system to assess the safety and efficacy of BAANs in mitigating CoV2 pathology in primary 3D human epithelial cells. Taken together, these data have led to the BANN as a therapy against CoV2 and other coronaviruses.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between. Now that the invention has been described,

What is claimed is:

1. A multi-functional broad-spectrum antiviral and anti-inflammatory nanosystem comprising:
at least one nanoparticle surface coated with at least one targeting moiety to form a targeted nanoparticle; and
at least one therapeutic agent encapsulated within the at least one targeted nanoparticle;
wherein the targeting moiety targets a coronavirus.

2. The nanosystem of claim 1, wherein the coronavirus is severe acute respiratory syndrome coronavirus 2 (SARS CoV-2) virus.

3. The nanosystem of claim 2, wherein the targeting moiety is a fatty acid selected from the group consisting of linoleic acid (LLA), linolenic acid (LNA), oleic acid, and lauric acid.

4. The nanosystem of claim 3, wherein the at least one therapeutic agent is selected from peroxisome proliferator activated receptor gamma (PPAR-γ) agonists, siRNA targeting conserved regions of the SARS CoV-2 virus, and combinations thereof.

5. The nanosystem of claim 4, wherein the PPAR-γ agonist is leriglitazone (LG) or pioglitazone (PG).

6. The nanosystem of claim 5, wherein the siRNA is siUTR, siLPro, or siMPro.

7. A method of treating a coronavirus infection in a patient in need thereof comprising:
administering to the patient in need thereof a therapeutically effective amount of a composition comprising
at least one nanoparticle surface coated with at least one targeting moiety to form a targeted nanoparticle;
at least one therapeutic agent encapsulated within the at least one targeted nanoparticle;
wherein the at least one therapeutic agent is a peroxisome proliferator activated receptor gamma (PPAR-γ) agonist, at least one siRNA targeting a conserved sequence in the coronavirus, or combinations thereof; and
a pharmaceutically acceptable carrier;
wherein the administration of the composition reduces viral replication in virus-infected cells of the patient.

8. The method of claim 7, wherein the coronavirus is severe acute respiratory syndrome coronavirus 2 (SARS CoV-2) virus.

9. The method of claim 8, wherein the targeting moiety targets the SARS CoV-2 virus and is a fatty acid selected from the group consisting of linoleic acid (LLA), linolenic acid (LNA), oleic acid, and lauric acid.

10. The method of claim 9, wherein the PPAR-γ agonist is leriglitazone (LG) or pioglitazone (PG).

11. The method of claim 10, wherein the siRNA is siUTR, siLPro, or siMPro.

12. The method of claim 11, wherein the at least one therapeutic agent is a combination of LG and at least one siRNA.

13. The method of claim 7, wherein the composition is administered to the patient intranasally.

14. A method of preventing a coronavirus severe acute respiratory syndrome coronavirus 2 (SARS CoV-2) infection in a patient in need thereof comprising:
prophylactically administering to the patient in need thereof a therapeutically effective amount of a composition comprising
at least one nanoparticle surface coated with at least one targeting moiety to form a targeted nanoparticle;
at least one therapeutic agent encapsulated within the at least one targeted nanoparticle;
wherein the at least one therapeutic agent is a peroxisome proliferator activated receptor gamma (PPAR-γ) agonist, at least one siRNA targeting a conserved sequence in the coronavirus, or combinations thereof; and
a pharmaceutically acceptable carrier;
wherein the prophylactic administration of the composition to the patient in need thereof inhibits the coronavirus infection.

15. The method of claim 14, wherein the coronavirus is severe acute respiratory syndrome coronavirus 2 (SARS CoV-2) virus.

16. The method of claim 15, wherein the targeting moiety targets the severe acute respiratory syndrome coronavirus 2 (SARS CoV-2) virus.

17. The method of claim 16, wherein the targeting moiety is a fatty acid selected from the group consisting of linoleic acid (LLA), linolenic acid (LNA), oleic acid, and lauric acid.

18. The method of claim 17, wherein the PPAR-γ agonist is leriglitazone (LG) or pioglitazone (PG).

19. The method of claim 18, wherein the at least one therapeutic agent is a combination of LG and at least one siRNA selected from siUTR, siLPro, or siMPro.

20. The method of claim 14, wherein the composition is administered intranasally.
